(19) United States
(12) Patent Application Publication
(10) Pub. No.: US 2023/0310538 A1
(43) Pub. Date: Oct. 5, 2023

(54) TISSUE REPAIR AND WOUND HEALING COMPOSITION AND METHOD USING LOW MOLECULAR WEIGHT HYDROLYZED COLLAGEN

(71) Applicant: GEORGE D. PETITO, BETHLEHEM, PA (US)

(72) Inventor: GEORGE D. PETITO, BETHLEHEM, PA (US)

(21) Appl. No.: 18/128,858

(22) Filed: Mar. 30, 2023

Related U.S. Application Data
(60) Provisional application No. 63/325,260, filed on Mar. 30, 2022.

(51) Int. Cl. A61K 38/01; A61K 45/06

(52) U.S. Cl. CPC A61K 38/014; A61K 45/06

(57) ABSTRACT

A tissue repair and wound healing composition with low molecular weight hydrolyzed collagen is provided. The wound healing composition with low molecular weight hydrolyzed collagen is a medicinal composition for facilitating the growth, protection and healing of tissues and cells in animals and humans. The main ingredient of the formulated composition is hydrolyzed collagen having a molecular weight of less than 1,000 Da, which can be combined with polysulfated glycosaminoglycans, hyaluronic acid or salts thereof, or a glucosamine salt, and mixtures thereof. The composition may include hydrolyzed collagen from multiple sources, such as bovine sourced hydrolyzed collagen or marine sourced hydrolyzed collagen and may further include hydrolyzed whey.

### CROSS-REFERENCE TO RELATED APPLICATION

This application claims the benefit of U.S. Provisional Patent Application No. 63/325,260, filed on Mar. 30, 2022.

### BACKGROUND

### 1. Field

A wound healing composition and method using low molecular weight hydrolyzed collagen relates to medical and nutritional compositions, and particularly, to a method and composition using hydrolyzed collagen having a molecular weight of less than 1,000 Daltons (Da).

### 2. Description of the Related Art

Just as nature has provided the skin as a barrier for protection, it has also provided mechanisms for skin repair. Depending upon the nature of the injury, this repair process may take hours, days, months, or even years. Many factors determine the length of time it takes for an injured skin to heal. Pathogenic contaminants may enter the body through the wound until the skin's integrity is restored. For this reason, it desirable to heal open wounds as quickly as possible.

Open wounds in the skin are a potential gateway for infectious or contaminating material to enter the body. The skin is a protective barrier to external contaminants. When the skin is damaged with an open breach, these contaminants are free to enter the body. Once inside the body, these contaminants may have effects of varying degrees, but almost always become more difficult to treat, and consequently slow the healing process of the original wound.

To fight infection, wound management traditionally involves an initial cleansing of the affected area to remove any contaminants such as dirt, clothing particles, or other debris. Damaged tissues and foreign materials are removed when necessary, and antiseptic agents are applied to sterilize the injured area. Sterile dressings are often applied, and periodically changed, to keep the injured area as clean and sterile as possible. Complex biological mechanisms occur during the healing process such as chemical signals attracting fibroblast cells to the wound site which ultimately generate connective structures mainly of collagen. Endothelial cells generate new blood capillaries that nurture new growth. The cell growth continues until the open wound is filled by forming permanent new tissue.

Because shortened periods of healing result in shortened exposure time, it would be beneficial to have any open wound heal as quickly as possible.

Traditional methods of wound healing have disadvantages, such as incomplete pigment removal, non-selective tissue destruction, and unsatisfactory cosmetic results, such as atrophic or hypertrophic scarring.

Thus, a wound healing composition and method solving the aforementioned problems is desired.

### SUMMARY

The wound healing composition and method using low molecular weight hydrolyzed collagen facilitates healing of damaged tissues, promoting tissue and cell growth, protecting cells and tissues, and reducing scar tissue. The composition includes proteinaceous amino acids, such as native collagen and/or hydrolyzed collagen. In some embodiments, hydrolyzed collagen may be combined with native collagen, further sources of amino acids, and/or at least one other therapeutic agent. For example, further sources of amino acids may include gelatins, whey, or hydrolyzed whey and the therapeutic agent may include glycosaminoglycans (e.g., polysulfated glycosaminoglycan), a glucosamine salt, or mixtures thereof.

The present compositions comprise hydrolyzed collagen, which not only serves as the main therapeutic component, but acts as a pharmaceutical carrier when preferably combined with a medicinal agent selected from the group consisting of hyaluronic acid and salts thereof, a polysulfated glycosaminoglycan, a glucosamine salt, and mixtures thereof to aid tissue and cells to grow and wounds to heal as quickly as possible.

In an embodiment, the hydrolyzed collagen in the composition is entirely or partially low molecular weight collagen. The low molecular weight collagen may comprise collagen having a molecular weight of less than 1,000 Da, less than 500 Da, or less than 400 Da. In an embodiment, the LMW hydrolyzed collagen may comprise hydrolyzed collagen having a molecular weight ranging from 10 Da to 1,000 Da. In an embodiment, the low molecular weight collagen may comprise collagen having a molecular weight of between 1,000 Da and 100 Da, between 500 Da and 100 Da, or between 400 Da and 100 Da. In a further embodiment, the low molecular weight collagen may consist of collagen having a molecular weight of less than 1,000 Da, less than 500 Da, or less than 400 Da. In a further embodiment, the low molecular weight collagen may consist of collagen having a molecular weight of between 1,000 Da and 100 Da, between 500 Da and 100 Da, or between 400 Da and 100 Da.

These and other features of the present subject matter will become readily apparent upon further review of the following specification.

### DETAILED DESCRIPTION OF THE PREFERRED EMBODIMENTS

### Definitions

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

The term “optional” or “optionally” means that the subsequently described event or circumstance may or may not occur, and that the description includes instances where said event or circumstance occurs and instances in which it does not.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

As used herein, “hydrolyzed collagen” is defined as a collagen hydrolysate polypeptide having a molecular weight lower than native collagen, i.e., in the 10 to 300,000 Daltons range, and is derived by hydrolysis.

As used herein, “hyaluronic acid” (HA) is rapidly hydrolyzed upon contact with treated tissue surfaces to monosaccharides, glucuronic acid and N-acetyl glucosamine. Chemical binding is enhanced with the use of hydrolyzed collagen, i.e., it is chemotactic. Hyaluronic acid can be used via injection into a joint for its anti-inflammatory effect to relieve pain and suffering. This curative effect is inherently terminated when hyaluronic acid is consumed by the healing body.

As used herein, “glycosaminoglycans” (GAGs) are polysaccharides found in vertebrate and invertebrate animals. Several GAGs have been found in tissues and fluids of vertebrate animals. The known GAGs are chondroitin sulfate, keratin sulfate, dermatan sulfate, hyaluronic acid, heparin, and heparin sulfate. GAGs and collagen are the major structural elements of all animal tissue. Their synthesis is essential for proper repair, treatment, protection, and maintenance of all tissues.

As used herein, “chondroitin sulfate”, a polysulfated GAG, is a linear polymer occurring in several isomers, named for the location of the sulfate group. Chondroitin-4 sulfate is found in nasal and tracheal cartilages of bovines and porcine. It is also found in the bones, flesh, blood, skin, umbilical cord, and urine of these animals. Chondroitin-6 sulfate has been isolated from the skin, umbilical cord, and cardiac valves of the aforementioned animals. Chondroitin-6 sulfate has the same composition, but slightly different physical properties from the chondroitin-4 sulfate. These are the most common isomers used in the present invention. The polymers are also known as polysulfated glycosaminoglycans (PSGAGs), chondroitin polysulfate sodium, chondrin, sodium chondroitin polysulfate, and sodium chondroitin sulfate. For consistency, the term, “chondroitin sulfate”, will be recited for all chondroitin sulfate isomers throughout this specification. Chondroitin sulfate is involved in the binding of collagen and is also directly involved in the retention of moisture in the tissue. These are both valuable chemical properties that aid the healing process.

As used herein, “subject” may refer to any animal, including but not limited to human beings and other mammals.

As used herein, “patient” may refer to a subject in need of treatment of a condition, disorder, or disease, such as an inflammatory condition or an immunological disorder.

### Wound Healing Composition

The wound healing composition includes proteinaceous amino acids, such as native collagen and/or hydrolyzed collagen, which may facilitate tissue and cell growth, as well as wound healing. Hydrolyzed collagen is a collagen hydrolysate polypeptide having a molecular weight lower than native collagen. Hydrolyzed collagen may be obtained by hydrolysis of native collagen. This may be accomplished by one of four methods: (1) alkaline hydrolysis; (2) enzymatic hydrolysis; (3) acid hydrolysis; and (4) synthetically, by fermentation. Any of these methods can be used to derive the hydrolyzed collagen from a collagen source.

In addition to native collagen and/or hydrolyzed collagen, the proteinaceous amino acids can include whey, whey isolates, and/or hydrolyzed whey. The native collagen and the hydrolyzed collagen can be derived from any suitable collagen source. The collagen source can be, for example, a bovine (skin and tendon preferred), a porcine, a reptile, a marine, an avian, or a synthetic source. The collagen can be derived from a single collagen source or a combination of collagen sources. The marine source can include any fish. The types of amino acid constituents and their sequences determine the beneficial healing qualities of hydrolyzed collagen. Hydroxylysine and hydroxyproline are amino acids found only in collagen and in no other medical protein hydrolysates. Hydroxylysine is typically found in concentrations from 0.7 to 1.2 wt. % in hydrolyzed collagen.

The native collagen and/or hydrolyzed collagen can be derived from bovine, porcine, chicken, and/or marine sources, for example. Bovine and porcine hydrolyzed collagen have high glycine, proline, hydroxyproline, and glutamic acid content. They also display hydrophilic properties. Bovine hydrolyzed collagen, for example, demonstrates strong hydrophilic properties and when used to treat wound sites demonstrates increased perfusion and epithelialization and decreased inflammatory reaction. In contrast, marine derived, i.e., marine sourced, hydrolyzed collagen has a different amino acid profile, with higher levels of aspartic acid, cysteine, glutamine, citrulline, and asparagine.

While hydrolyzed collagen of any molecular weight may be used, the hydrolyzed collagen can be Low Molecular Weight hydrolyzed collagen (hereinafter “LMW hydrolyzed collagen”) having a molecular weight less than 1,000 Da. The LMW hydrolyzed collagen can have increased bioavailability. The LMW hydrolyzed collagen may comprise hydrolyzed collagen having a molecular weight of less than 1,000 Da, less than 500 Da, or less than 400 Da. In an embodiment, the LMW hydrolyzed collagen may comprise hydrolyzed collagen having a molecular weight ranging from 10 Da to 1,000 Da. For example, the LMW hydrolyzed collagen may comprise hydrolyzed collagen having a molecular weight of between 1,000 Da and 100 Da, between 500 Da and 100 Da, or between 400 Da and 100 Da. In a further embodiment, the LMW hydrolyzed collagen may consist of hydrolyzed collagen having a molecular weight of less than 1,000 Da, less than 500 Da, or less than 400 Da. In a further embodiment, the LMW hydrolyzed collagen may consist of hydrolyzed collagen having a molecular weight of between 1,000 Da and 100 Da, between 500 Da and 100 Da, or between 400 Da and 100 Da.

The LMW hydrolyzed collagen can be prepared by partially hydrolyzing native collagen in any suitable manner known in the art. Preferably, raw materials from one or more collagen sources are ground to a powder, enzymatically treated, fractionated, and purified to obtain high molecular weight hydrolyzed collagen. Bulk fractionation methods known in the art can be used. The raw materials can include, for example, fat, blood, tissue, and/or bone marrow from one or more collagen sources. Raw material from fish can further include, e.g., fish head and/or fins.

The present composition may include a combination of hydrolyzed collagen and native collagen. Combining native collagen with hydrolyzed collagen may enhance the bacteriostatic effects, as well as the cellular repair and wound healing properties of the composition. The different molecular weights of the native collagen and the hydrolyzed collagen in the composition may facilitate better control over absorption amount and absorption time of the composition, as well as the degradation time of the composition. For example, by varying the ratio of the native collagen to hydrolyzed collagen, various absorption rates and degradation rates may be achieved. Preferably, the composition includes a combination of LMW hydrolyzed collagen and native collagen. Soluble and/or insoluble native collagen may be used.

The composition may include about 1% by weight to about 99% by weight hydrolyzed collagen. For example, the composition may include about 10% by weight to about 85% by weight hydrolyzed collagen or about 20% by weight to about 75% by weight hydrolyzed collagen, or about 30% by weight to about 65% by weight hydrolyzed collagen. The hydrolyzed collagen is preferably LMW hydrolyzed collagen. The composition may include about 0.1% by weight to about 65% by weight of soluble or insoluble native collagen. For example, the composition may include about 2% by weight to about 45% by weight of soluble or insoluble native collagen, or about 10% by weight to about 30% by weight of soluble or insoluble native collagen. The composition may include hydrolyzed collagen cross-linked with native collagen. For example, the composition may include about 0.1% by weight to about 65% by weight insoluble or soluble native collagen crosslinked with LMW hydrolyzed collagen. Other amounts below and above these ranges may be used.

In an embodiment, the composition can include mixtures of collagen from different collagen sources. For example, the composition can include bovine sourced collagen, marine sourced collagen, chicken sourced collagen, and whey protein. Alternatively, the composition can include bovine sourced collagen and marine sourced collagen. According to one embodiment, the proteinaceous amino acids in the composition can include bovine sourced hydrolyzed collagen, marine sourced hydrolyzed collagen, and hydrolyzed whey protein. The composition can further include elastin. Hydrolyzed whey protein offers another alternative amino acid profile, rich in glutamic acid, isoleucine, leucine, threonine, tyrosine, and valine.

Varying the source of the amino acids in the composition can control the chemotactic, hydrophilic, and cell proliferative properties of the composition. These properties may be manipulated in order to optimize the wound healing process. This optimization may adjust the timing and balance of stimulating the inflammatory and vascular systems, as well as involvement of connective tissues and epithelial cells.

For example, a heavily exudative or wet wound can be treated, at least initially, with a highly hydrophilic composition, including at least about 50% by weight bovine sourced hydrolyzed collagen (e.g., about 50% to about 60% by weight bovine sourced hydrolyzed collagen), at least about 20% by weight marine sourced hydrolyzed collagen (e.g., about 20% to about 30% by weight marine sourced hydrolyzed collagen), and up to about 30% by weight hydrolyzed whey protein (e.g., about 5% to about 30% by weight hydrolyzed whey protein). Elastin may be added to this composition (e.g., up to about 20%) during the closing phase of wound treatment to improve tensile strength and reduce scar formation. In contrast, a dry wound can be treated with at least about 50% by weight marine sourced hydrolyzed collagen, about 20% by weight or less hydrolyzed whey, about 10% by weight or less bovine sourced hydrolyzed collagen, and about 5% by weight or less elastin.

In a further embodiment, the composition may comprise bovine sourced hydrolyzed collagen, marine sourced hydrolyzed collagen and hydrolyzed whey. The composition may include about 70% bovine sourced hydrolyzed collagen, about 20% marine sourced hydrolyzed collagen, and about 10% bovine derived hydrolyzed whey. Some or all of the hydrolyzed collagen in this composition may be LMW hydrolyzed collagen.

One or more additional therapeutic agents may be included in the composition to further speed the healing process, decrease scarring and increase tissue strength. Examples of suitable therapeutic agents that may be combined with the hydrolyzed collagen are glycosaminoglycans (GAGs), particularly GAGs useful for cellular repair. Antimicrobials may also be included in the composition to further enhance its bacteriostatic quality, as can antibiotics (such as tetracycline, streptomycin, and cephalosporin) and antibacterials (such as iodine, parachlorometaxylenol, and chlorhexidine gluconate or acetate). The composition may further include lipoic acid, alpha lipoic acid, one or more vitamins (e.g., vitamin A, vitamin B12, vitamin C, vitamin E), omega compounds or omega-3 fatty acid compounds (e.g., α-linolenic acid “ALA”, Eicosapentaenoic acid “EPA”, Docosahexaenoic acid “DHA”),), antioxidants (e.g., superoxide dismustase, glutathione peroxidase, glutathione reductase), and/or phytochemicals (e.g., zeaxanthin, lutein). Also, it has been established that hydrolyzed collagen used as a carrier in powder form, paste or a lyophilized foam has hemostatic qualities when combined with thrombin to improve healing of wounds.

Hydrolyzed collagen in combination with GAGs, specifically a PSGAG (such as chondroitin sulfate), can be useful for the prevention and treatment of wound diseases. The hydrolyzed collagen combines with a PSGAG to bond or adhere selectively to tissue, resulting in interference with and/or displacement of bacterial or other infectious agents. In addition, the combination product may exhibit anti-enzyme activity or the ability to inhibit enzyme activity.

The hydrolyzed collagen accelerates the healing process by allowing an injured tissue to repair itself by producing and remodeling more collagen and other proteoglycans (PGs). The building blocks for collagen production are the amino acids found in hydrolyzed collagen. Hyaluronic acid and other proteoglycans (PGs) provide the framework for collagen production to follow. The PGs hold water to provide an excellent environment for healing of the tissue to begin. When in the wound site, any unused collagen that was produced is simply degraded to the amino acid. The rate-limiting step in the production of collagen is the conversion of glucose to glucosamine for the production of hyaluronic acid and other glycosaminoglycans (GAGs).

The composition can include one or more therapeutic agents, such as an antibiotic, and/or one or more additives, such as glutamine, glycosaminoglycans, zinc, alginates, cellulose, bioactive glass, and/or honey.

These are simplified examples, as wound healing is complex and wound specific. More complicated wounds, such as diabetic wounds, are treated using customized treatment regimens. For example, a diabetic wound can initially be treated as a wet wound, but with significantly more emphasis on hydrolyzed whey in the early treatment composition. During the later closing phase of wound treatment, the composition can be shifted to up to about 40% by weight marine sourced hydrolyzed collagen, up to about 25% by weight bovine sourced hydrolyzed collagen, and up to about 5% by weight elastin.

Further examples of this wound healing composition optimized for different applications include: about 70% bovine sourced hydrolyzed collagen and about 30% marine sourced hydrolyzed collagen; about 50% bovine sourced hydrolyzed collagen, about 30% marine sourced hydrolyzed collagen, and about 20% hydrolyzed whey; about 40% bovine sourced hydrolyzed collagen, about 20% marine sourced hydrolyzed collagen, about 20% hydrolyzed whey, and about 20% elastin; about 20% bovine sourced hydrolyzed collagen, about 40% marine sourced hydrolyzed collagen, about 20% hydrolyzed whey, and about 20% elastin; about 20% bovine sourced hydrolyzed collagen, about 40% marine sourced hydrolyzed collagen, and about 40% hydrolyzed whey; and about 30% bovine sourced hydrolyzed collagen, about 30% marine sourced hydrolyzed collagen, about 30% hydrolyzed whey, and about 10% elastin. The bovine hydrolyzed collagen can have a molecular weight of about 500 Daltons to about 10,000 Daltons. The porcine hydrolyzed collagen can have a molecular weight of about 1,000 Daltons to about 15,000 Daltons. The salmon hydrolyzed collagen can have a molecular weight of about 100 Daltons to about 10,000 Daltons. The elastin can have a molecular weight of about 35,000 Daltons to about 145,000 Daltons.

The composition may be used to heal topical and/or internal wound sites. For example, the composition may be used prior to and after surgery to minimize cell damage and to expedite wound healing. The composition may be useful during surgery to foster separation of tissue to prevent adhesion formation. The composition may be used as a filler for a wound site and remain in the wound site as it heals, becoming part of the granulated tissue.

The composition may be useful for applications relating to cosmetic and plastic surgery, e.g., as a filler for lines and wrinkles formed in the skin.

The composition may take a physical form used in topical administration, such as a gel, spray, powder, paste, foam, film for incorporation in a dressing bandage, or a topically applied patch. The composition may take a physical form used in internal administration, such as an injectable liquid or an orally ingestible liquid.

The composition may be formulated for use as a medical food. Medical foods are foods that are formulated to be consumed or administered under supervision of a physician and which are intended for the specific dietary management of a disease condition for which distinctive nutritional requirements are established by medical evaluation. The composition formulated for use as a medical food may be formulated for oral consumption or for tube feeding.

The powder composition will preferably have a moisture content of about 2-10 wt. % and a pH range of 5.5 to 6.5. The powder composition will have an ash content of less than 2.5 wt. % and an isotonic point of 5.0 to 6.5. In use, the powder composition may be the preferred physical form for use with irregularly shaped wounds. Tunnel wounds, flaps, and other non-conformative sites may be managed with the powder composition because it easily conforms to any shape wound and may be applied by a poofer bottle or otherwise blown into difficult to reach wound sites. The powder is especially useful in wounds having a large amount of exudate, as the powder can absorb nearly 30 times its own weight. As the powder absorbs the exudate, a gel is formed, which completely fills the wound site, forming a mechanical barrier against bacterial infection. The powder does not exhibit the characteristic fly-away when being applied to the wound site, and administration is perfected due to the precise powder placement.

The gel form of the composition is especially useful in wounds with lesser amounts of exudate, in burns, and in surgical sites. Application of the gel can be dispensed through a tube, a syringe, or the reservoir in a topical patch. The gel can be made of about 1-75 wt. % hydrolyzed collagen and 1-99 wt. % water. It is preferable to use about 60 wt. % collagen. The gel is formed by adding purified water, e.g., sterile water, to the powder. The gel has the added advantage of adding moisture to the wound site, as well as inherent bacteriostatic properties, and stays positioned where applied.

A film form of the medicament composition may be made by mixing the powdered form with deionized water under heat at 155-175° F. Cross-linking and other agents, such as humectant, propylene glycol, sorbitol, and glycerine, may be added to the mixture. A preservative (such as benzyl alcohol or paraben) can be added. The mixture is cast on a belt liner by knife on a roll coating machine to form a liquid film, which is oven-dried. The film form can also be formed by cooling the liquid solution. These films can be used for drug or other chemical delivery, especially in dental applications. Antimicrobial and other medicinal agents can also be added to the film as needed for specific applications.

The composition may be formulated as a nutritional supplement. In an embodiment, the nutritional supplement can include at least suitable nutritional additive. For example, at least one of vitamin A, vitamin C, vitamin E, vitamin B12, magnesium oxide, chelated manganese, grape seed extract, zinc, an alginate, cellulose, honey, chromium picolinate, selenium, glutamine, alpha lipoic acid, Coenzyme Q10, and glycosaminoglycans can be added to the composition to produce a nutrient composition for oral intake.

The present compositions may also be administered as compositions prepared as foods for humans or animals, including medical foods, functional foods, special nutrition foods and dietary supplements. A “medical food” is a product prescribed by a physician that is intended for the specific dietary management of a disorder or health condition for which distinctive nutritional requirements exist and may include formulations fed through a feeding tube (referred to as enteral administration or gavage administration) or via intravenous injection (i.v.).

These medical foods may be formulated according to any method known in the art. For example, methods of formulating medical foods are discussed in U.S. Pat. Nos. 5,637,324 A and 6,210,701 B1, the contents of which are incorporated herein by reference.

A “dietary supplement” shall mean a product that is intended to supplement the human diet and may be provided in the form of a pill, capsule, tablet, or like formulation. By way of non-limiting example, a dietary supplement may include one or more of the following dietary ingredients: vitamins, minerals, herbs, botanicals, amino acids, and dietary substances intended to supplement the diet by increasing total dietary intake, or a concentrate, metabolite, constituent, extract, or combinations of these ingredients, not intended as a conventional food or as the sole item of a meal or diet. Dietary supplements may also be incorporated into foodstuffs, such as functional foods designed to promote control of glucose levels. A “functional food” is an ordinary food that has one or more components or ingredients incorporated into it to give a specific medical or physiological benefit, other than a purely nutritional effect. “Special nutrition food” means ingredients designed for a particular diet related to conditions or to support treatment of nutritional deficiencies.

When formulated as a dietary supplement, in addition to collagen the compositions may include one or more of a vitamin, a glycosaminoglycan, alpha lipoic acid, a berry extract, and berberine. It is specifically contemplated that the dietary supplements disclosed herein may use liposomes as a delivery vehicle. In the alternative, the compositions may include one or more polymeric molecules and or a biopolymer. In certain embodiments the compositions may be formulated as films or as film-forming compositions.

The present compositions may be formulated for topical cosmetic applications, such as for anti-aging or anti-wrinkle applications. In such applications the compositions may include further ingredients, including one or more of an emulsifier, preservative, thickener, emollient, pigment, glimmer, fragrance, paraben, or a combination thereof. These compositions may be formulated by techniques generally known in the art, including those discussed in U.S. Pat. No. 10,905,640 B2, U.S. Pat. No. 6,524,459 B2, and US 2002/0058053 A1, the contents of which are incorporated herein by reference.

The composition can be used as an excellent drug vehicle system including acidic, neutral or complexed drug medications.

### Methods of Wound Healing

The method of treating a wound may include administering at least one of the compositions disclosed above to a subject in need thereof.

In an embodiment, subjects having heavily exudative or wet wounds can be treated, at least initially, with a highly hydrophilic composition, including at least about 50% by weight bovine sourced hydrolyzed collagen (e.g., about 50% to about 60% by weight bovine sourced hydrolyzed collagen), at least about 20% by weight marine sourced hydrolyzed collagen (e.g., about 20% to about 30% by weight marine sourced hydrolyzed collagen), and up to about 30% by weight hydrolyzed whey protein (e.g., about 5% to about 30% by weight hydrolyzed whey protein). Elastin may be added to this composition (e.g., up to about 20%) during the closing phase of wound treatment to improve tensile strength and reduce scar formation. Some or all of the hydrolyzed collagen in this composition may be LMW hydrolyzed collagen.

In a further embodiment, subjects having dry wounds can be treated with a composition including at least about 50% by weight marine sourced hydrolyzed collagen, about 20% by weight or less hydrolyzed whey, about 10% by weight or less bovine sourced hydrolyzed collagen, and about 5% by weight or less elastin. Some or all of the hydrolyzed collagen in this composition may be LMW hydrolyzed collagen.

In a further embodiment, subjects may be administered a composition including bovine sourced hydrolyzed collagen, marine sourced hydrolyzed collagen and hydrolyzed whey. The composition may further include about 70% bovine sourced hydrolyzed collagen, about 20% marine sourced hydrolyzed collagen, and about 10% bovine derived hydrolyzed whey. Some or all of the hydrolyzed collagen in this composition may be LMW hydrolyzed collagen.

In a further embodiment, a composition including bovine sourced hydrolyzed collagen, marine sourced hydrolyzed collagen and hydrolyzed whey may be administered as a medical food to a subject in need thereof. The composition may include about 70% bovine sourced hydrolyzed collagen, about 20% marine sourced hydrolyzed collagen, and about 10% bovine derived hydrolyzed whey. Some or all of the hydrolyzed collagen in this composition may be LMW hydrolyzed collagen.

### Other Medical Uses

In some embodiments, the compositions disclosed herein may be used in combination with other active agents, including drugs, vitamins, glucosamine, glycosaminoglycans, other collagens, honey, aloe, and anesthetic agents including benzocaine, lidocaine, or the like. When used in a combination treatment, the collagen composition may act as a carrier and may improve the bioavailability, absorption, stability, or skin penetration of the other active agent.

In further embodiments, the compositions disclosed herein may be used as part of a filler treatment, including but not limited to use as a line filler restorer, or in compositions assisting in reducing the healing time of surgical closures or other internal wounds.

In some embodiments, the compositions disclosed herein may be used as soluble collagen injections, solid constructs reconstituted from solution, decellularized collagen matrices, or for treatment of chronic wounds, burns, venous or diabetic ulcers, or in plastic, reconstructive, cardiovascular, bone, cartilage, or general surgery, or generally in the practice of urology, proctology, gynecology, ophthalmology, otolaryngology, neurosurgery, dentistry, or cosmetology.

In some embodiments, the compositions disclosed herein may be used for cosmetic applications. The compositions disclosed herein may be useful additives in products intended to improve moisture retention, reduce the appearance of wrinkles, or the like. Further, the compositions disclosed herein may be formulated as topical cosmetic treatments (creams and the like), or as injectable compositions (for treatment of wrinkles and the like). When formulated for cosmetic applications, the compositions disclosed herein may further act as a carrier for other active agents, including but not limited to hyaluronic acid and the like.

It is to be understood that the wound healing composition and method of using low molecular weight hydrolyzed collagen is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

We claim:

1. A composition comprising low molecular weight hydrolyzed collagen, wherein the low molecular weight hydrolyzed collagen has a molecular weight of about 10-1,000 Daltons.

2. The composition of claim 1, further comprising bovine sourced hydrolyzed collagen, marine sourced hydrolyzed collagen, and hydrolyzed whey.

3. The composition of claim 2, wherein the bovine sourced hydrolyzed collagen comprises low molecular weight hydrolyzed collagen having a molecular weight of about 10-1,000 Daltons.

4. The composition of claim 2, wherein the marine sourced hydrolyzed collagen comprises low molecular weight hydrolyzed collagen having a molecular weight of about 10-1,000 Daltons.

5. The composition of claim 2, wherein the composition comprises about 70% by weight bovine sourced hydrolyzed collagen, about 20% by weight marine sourced hydrolyzed collagen, and about 10% by weight hydrolyzed whey.

6. The composition of claim 1, further comprising at least one therapeutic agent selected from the group consisting of glutamine, glycosaminoglycans, zinc, alginates, cellulose, bioactive glass, and/or honey.

7. A medical food comprising the composition of claim 1.

8. A nutritional supplement comprising the composition of claim 1.

9. A cosmetic comprising the composition of claim 1.

10. The composition of claim 1, further comprising an active agent selected from the group consisting of drugs, vitamins, glucosamine, glycosaminoglycans, native collagen, honey, aloe, benzocaine, and lidocaine.

11. A method of treating a wound comprising administering the composition of claim 1 to a subject in need thereof.

12. A method of delivering the active agent to a subject in need thereof, comprising administering the composition of claim 10 to the subject.

13. A method of reducing wrinkles, comprising topical administration of the composition of claim 1 to a subject in need thereof.

14. A method of reducing photoaging of skin, comprising topical administration of the composition of claim 1 to a subject in need thereof.

15. A method of healing internal wounds, comprising administering the composition of claim 1 to a subject in need thereof.

16. The method of claim 15, wherein the method further comprises reducing the recovery time from a surgical procedure.
